(19) United States
(12) Patent Application Publication
(10) Pub. No.: US 2015/0320816 A1
(43) Pub. Date: Nov. 12, 2015

(54) COMPOSITIONS FOR PROMOTION OF WOUND HEALING AND/OR SCAR REDUCTION

(71) Applicant: Trinity Pharma Group, New Port Richey, FL (US)

(72) Inventors: Devan Patel, Tampa, FL (US); Krutika Patel, Tampa, FL (US)

(73) Assignee: Trinity Pharma Group, New Port Richey, FL (US)

(21) Appl. No.: 14/705,587

(22) Filed: May 6, 2015

Related U.S. Application Data
(60) Provisional application No. 61/988,977, filed on May 6, 2014.

(51) Int. Cl. A61K 36/48; A61K 31/215; A61K 31/4422; A61K 31/167; A61K 45/06; A61K 31/01; A61K 31/495; A61K 31/522; A61K 31/195; A61K 31/196; A61K 31/355; A61K 35/35; A61K 31/60; A61K 31/203; A61K 31/569; A61K 31/4745; A61K 9/00; A61K 31/4166

(52) U.S. Cl. CPC A61K 36/48; A61K 31/4166; A61K 31/215; A61K 31/4422; A61K 31/167; A61K 45/06; A61K 31/01; A61K 31/495; A61K 31/522; A61K 31/195; A61K 31/196; A61K 31/355; A61K 35/35; A61K 31/60; A61K 31/203; A61K 31/569; A61K 31/4745; A61K 9/0014

(57) ABSTRACT

The present invention relates to a wound healing and scar reduction composition that is a unique product with key ingredients carefully selected to optimize scar reduction. The composition when used can help reduce the appearance of old and new scars. It is an ideal choice for use on all types of scar tissue including, new scars, old scars, surgical, keloids, hypertrophic, stretch marks and any skin condition that would benefit from barrier protection.

### CROSS REFERENCE TO RELATED APPLICATIONS

The present application claim priority to U.S. Provisional Application No. 61/988,977 filed May 6, 2014. The provisional application in incorporated by its entirety herein by reference.

### BACKGROUND OF THE INVENTION

The present invention is directed to the use of new compositions for the promotion of wound healing and/or scar reduction. The progress of a general scar through the four stages of evolution: inflammation, proliferation, Epithelization, and maturation. Prior to the development of the present composition, there were non-invasive treatment options for scars. The two main options were pressure therapy and silicone therapy. The two therapies were used in conjunction when needed. Pressure therapy was used primarily to control hypertrophic scars after burns by speeding up the formation of collagen thus flattening the scar. Pressure therapy method is more effective when used with the silicone therapy.

Silicone therapy has three different general paths of treatment. Silicone Gel Sheets are thick sheets of silicone that are taped to the body and have a 3 month treatment period. This method was the main course of treatment until recently. Silicone Gels are the current main form of silicone-based treatment for scars now. The application is simple and, in specific cases, can dry within minutes to allow other topical products to be applied to the skin. Silicone Sprays are the newest avenue for silicone therapy. It allows for application to larger areas and mainly used for burn patients as the scars are larger and can be painful to the touch.

A need therefore exists to provide a product that when used can assist in promoting wound healing and scar reduction.

### SUMMARY OF THE INVENTION

The present invention is directed to a wound healing and scar reduction composition that is a unique product with key ingredients. The ingredients are carefully selected to optimize scar reduction. The composition when used can help reduce the appearance of old and new scars. It is an ideal choice for use on all types of scar tissue including, new scars, old scars, surgical, keloids, hypertrophic, stretch marks and any skin condition that would benefit from barrier protection.

In one application there is provided a base compound in combination with the following active ingredients, Phenytoin, Misoprostol, Nifedipine, and Lidocaine. The active ingredients may be provided in the following percentages based on a finished composition: Phenytoin: 1-10%, Misoprostol: 0.001-0.003%, Nifedipine: 1-5%, and Lidocaine: 1-6%. In addition to the above, one or more of the following ingredients may be included: Gentamicin: 0.5-2.0%, Tobramicin: 0.5-2.0%, Ciprofloxacin: 0.5-3.0%, Levofloxacin: 0.5-5.0%, Vancomycin: 2-6%, Linezolid: 0.5-5.0%, Mupirocin: 1-5%, Clindamycin: 0.5-5.0%, Doxycycline: 0.5-5.0%, Amikacin: 0.5-3.0%, Cefuroxime: 1-3%, Meropenem: 0.5-3.0%, Metronidazole: 0.5-3.0%, Bacitracin: 100-1000 U/GM, Colistimethate: 0.5-3.0%, Ampicillin: 0.5-3.0%, and Tetracycline: 0.5-3.0%. Yet further to the above, one or more of the following ingredients may be provided: Itraconazole: 0.5-3.0%, Fluconazole: 0.5-3.0%, and Nystatin: 10,000-50,000 U/GM. Yet further still, one or more of the following ingredients may be provided: Xylitol: 0.5-2.0%, Rifampin: 0.5-5.0%, Collagen: 0.5-5.0%, Collagenase: 100-500 U/GM, Fluticasone: 0.1-2.0%, Betamethasone: 0.025-0.2%, and Urea: 5-50%. The above compositions may be provided on either Amazonian oil and Pracaxi oil, and may further include one or more of the following: Levocetirizine, Pentoxyifylline, Gabapentin, Tranilast, Vitamin E, Emu Oil, Salicylic Acid, Tretinoin (Retinoic Acid).

In another aspect of the invention there is provided a scar reduction composition, comprising: Fluticasone Propionate, Levocetirizine, Lidocaine. The active ingredients may be provided in the following percentages based on a finished composition: Fluticasone Propionate 1%, Levocetirizine 2%, Lidocaine 5%, in base gel for topical application to a scar and/or skin wound.

In another aspect of the invention there is provided a scar reduction composition, comprising: Imiquimod, Fluticasone Propionate, Levocetirizine, Lidocaine. The active ingredients may be provided in the following percentages based on a finished composition: Imiquimod 2.5%, Fluticasone Propionate 1%, Levocetirizine 2%, Lidocaine 5%, in a base gel for topical application to a scar.

In yet another aspect of the invention there is provided a scar reduction composition, comprising: Imiquimod, Betamethasone Dipropionate, Pentoxifylline. The active ingredients may be provided in the following percentages based on a finished composition: Imiquimod 2.5%, Betamethasone Dipropionate 0.1%, Pentoxifylline 3%, in a base gel for topical application to a scar.

### BRIEF DESCRIPTION OF THE FIGURES

FIGS. 1A through 1G are photos of a patient following the use of one of the compositions in accordance with the present invention; and

FIG. 2 is an illustration of a patient's wound when using a composition in accordance with one or more of the embodiments as shown through a course of treatment;

FIGS. 3A-3C illustrate a patient's wound when using a composition in accordance with one or more of the embodiments as shown through a course of treatment;

FIGS. 4A-4F illustrate a patient's wound when using a composition in accordance with one or more of the embodiments as shown through a course of treatment; and

FIGS. 5A-5B illustrate a patient's wound when using a composition in accordance with one or more of the embodiments as shown through a course of treatment.

### DETAILED DESCRIPTION OF THE INVENTION

The present invention is directed to compositions for wound healing and/or scar reduction. The compositions when used has shown to significantly help reduce the appearance of old and new scars. It is an ideal choice for use on all types of scar tissue including, new scars, old scars, surgical, keloids, hypertrophic, stretch marks and any skin condition that would benefit from barrier protection.

The composition is preferably provided in a gel and should be evenly applied and gently rubbed into the healed incision or scar twice daily or as directed by a doctor. The composition can be used as soon as the wound has healed and/or the sutures have been removed. Use may also start immediately on older scars. The composition is not intended for use on open wounds.

The composition in accordance with a first embodiment directed to a wound healing composition includes as a base compound the following ingredients: Phenytoin, Misoprostol, Nifedipine, and Lidocaine. Typical range of these ingredients as provided by the following amounts (where the percentage of taken from the finished compound): Phenytoin: 1-10%, Misoprostol: 0.001-0.003%, Nifedipine: 1-5%, and Lidocaine: 1-6%.

Lidocaine is a local anesthetic used to help treat pain associated during the healing process. Some scars can be associated with neuropathic pain and topical Lidocaine 5% has been shown to decrease this pain.

In additional to the base compound one or more of the following antibiotics may be added to the base compound, and as provided in the following amounts: Gentamicin: 0.5-2.0%, Tobramicin: 0.5-2.0%, Ciprofloxacin: 0.5-3.0%, Levofloxacin: 0.5-5.0%, Vancomycin: 2-6%, Linezolid: 0.5-5.0%, Mupirocin: 1-5%, Clindamycin: 0.5-5.0%, Doxycycline: 0.5-5.0%, Amikacin: 0.5-3.0%, Cefuroxime: 1-3%, Meropenem: 0.5-3.0%, Metronidazole: 0.5-3.0%, Bacitracin: 100-1000 U/GM, Colistimethate: 0.5-3.0%, Ampicillin: 0.5-3.0%, and Tetracycline: 0.5-3.0%.

The composition may also include one or more of the following antifungals alone or in combination with one or more antibiotics plus the base compounds: Itraconazole: 0.5-3.0%, Fluconazole: 0.5-3.0%, and Nystatin: 10,000-50,000 U/GM.

In addition, one or more of the following ancillary ingredients may be added to the base compounds, antibiotics, and/or antifungals: Xylitol: 0.5-2.0%, Rifampin: 0.5-5.0%, Collagen: 0.5-5.0%, Collagenase: 100-500 U/GM, Fluticasone: 0.1-2.0%, Betamethasone: 0.025-0.2%, and Urea: 5-50%. Fluticasone and Betamethasone are corticosteroids used to decrease the inflammation of new scars and help prevent the formation of a hypertrophic scar thus allowing optimal healing to occur. Corticosteroids such as Triamcinolone have been injected into keloid scars or post-surgical skin for the treatment or prevention of scar tissue formation. These injections can be painful and have to be done monthly for 2 to 3 months or longer depending on patient response.

Collagenase help contributes towards the formation of granulation tissue and subsequent epithelization. It acts as an active sterile enzymatic debriding agent to clean wounds and promotes an optimal environment and is effective in the removal of detritus.

In addition to the above, the composition be directed to reduce scars and may include Amazonian oil and Pracaxi oil, which works to improve the overall appearance, texture and color of the scars. The oils may be in a topical anhydrous silicone base such as PracaSil PIus™, manufactured by PCCA. In addition to these oils, additional ingredients may be provided in the scar reduction composition, such as but not limited to:

Levocetirizine is an antihistamine used to decrease the itching associated with healing skin. During the normal healing response mast cells play a vital role. It has been found that in the formation of some keloid and hypertrophic scars there is an abnormally high amount of mast cells involved. The mast cells can release histamine causing the healing skin to itch. Levocetririzine will decrease collagen production at higher topical concentrations. Pentoxyifylline, which is a blood viscosity reducer agent and acts to decreases collagen production. Gabapentin, which is an inhibition of excitatory neurotransmitters and decreases pain caused by firing neurons. Tranilast, which is an Anti-allergic and may be an indication for keloid and hypertrophic scar. Vitamin E, which promotes skin elasticity thus minimizing the tissue tearing. Emu Oil, which is derived from emu fat and claims to have anti-inflammatory and skin penetrating properties. Salicylic Acid, which is a peeling agent for the treatment of acne, as it removes intercellular lipids that are covalently linked to the cornified envelope surrounding cornified epitheliod cells. Tretinoin (Retinoic Acid), which is a Metabolite of vitamin A (retinol) and responsible for most of the activity of vitamin A and is used for acne treatment, anti-wrinkle, and stretch marks

In Composition 1.0 the composition is provided for post-surgical and new scars. The composition includes Fluticasone Propionate 1%, Levocetirizine 2%, Lidocaine 5%, all in a Pracasil base gel.

In another embodiment a composition is provided for reducing stretch marks. The composition includes: Levocetirizine 2%, Vitamin E Acetate 2%, Emu Oil 2%, all in Pracasil base gel.

In another embodiment a composition is provided for treating hypertrophic or Keloid Scars. The composition includes: Imiquimod 2.5%, Fluticasone Propionate 1%, Levocetirizine 2%, Lidocaine 5%, all in Pracasil base.

Imiquimod, which can be added to any of the scar formulations, can be used to treat hypertrophic scarring by increasing collagen breakdown.

In yet another embodiment, a composition is provided for treating scars. The composition includes: Imiquimod 2.5%, Betamethasone Dipropionate 0.1%, Pentoxifylline 3%, all in a SiloMac Gel.

Pentoxifylline, which can be added to any of the scar compositions, is a substituted methylxanthine that can inhibit fribroblast proliferation and extracellular collagen which can help to decrease hypertrophic scarring.

SiloMac Anhydrous gel contains silicone and Allium Cepa Bulb Extract which have been shown to aid in color, height, and itching of various scars.

Additional compositions provided herein may include:

In Composition 1.0, the composition includes the following ingredients provided for in a base for topical application to a patient: FLUTICASONE 1%+LEVOCETIRIZINE 2%+LIDOCAINE 5%.

In Composition 1.1A, the composition includes the following ingredients provided for in a base for topical application to a patient: BETAMETHASONE 0.1%+LEVOCETIRIZINE 2%+LIDOCAINE 5%+KETOPROFEN 5%.

In Composition 1.1C, the composition includes the following ingredients provided for in a base for topical application to a patient: BETAMETHASONE 0.1%+LEVOCETIRIZINE 2%+LIDOCAINE 5%.

In Composition 1.2, the composition includes the following ingredients provided for in a base for topical application to a patient: FLUTICASONE 1%+LEVOCETIRIZINE 2%+LIDOCAINE 5%+DICLOFENAC 3%.

In Composition 1.3, the composition includes the following ingredients provided for in a base for topical application to a patient: BETAMETHASONE 0.1%+LEVOCETIRIZINE 2%+DICLOFENAC 3%+LIDOCAINE 5%.

In Composition 1.4, the composition includes the following ingredients provided for in a base for topical application to a patient: FLUTICASONE 1%+LEVOCETIRIZINE 2%+LIDOCAINE 5%+COLLAGEN.ASE 350U+GM

In Composition 1.5, the composition includes the following ingredients provided for in a base for topical application to a patient: FLUTICASONE 1%+COQ101%+TRETINOIN 0.1%.

In Composition 1.6, the composition includes the following ingredients provided for in a base for topical application to a patient: SALICYLIC ACID 3%+RETINOIC ACID (TRETINOIN ACID) 0.1%+FLUTICASONE 1%.

In Composition 1.7, the composition includes the following ingredients provided for in a base for topical application to a patient: LEVOCETIRIZINE 2%+VITAMIN E ACETATE 2%+EMU OIL 2%.

In Composition 1.8, the composition includes the following ingredients provided for in a base for topical application to a patient: TRANILAST 1%+FLUTICASONE 1%+LEVOCERTIRIZINE 2%.

In Composition 1.9C, the composition includes the following ingredients provided for in a base for topical application to a patient: SALICYLIC ACID 3%+RETINOIC ACID (TRETINOIN ACID) 0.1%+BETAMETHASONE 0.1%.

In Composition 19.9, the composition includes the following ingredients provided for in a base for topical application to a patient: LEVOCETIRIZINE 2%+LIDOCAINE 5%.

In Composition 2.0C, the composition includes the following ingredients provided for in a base for topical application to a patient: TRANILAST 1%+BETAMETHASONE 0.1%+LEVOCERTIRIZINE 2%.

In Composition 2.1, the composition includes the following ingredients provided for in a base for topical application to a patient: BETAMETHASONE 0.1%+LEVOCETIRIZINE 2%+LIDOCAINE 5%+KETOPROFEN 5%+TRAMADOL 5%.

In Composition 2.2, the composition includes the following ingredients provided for in a base for topical application to a patient: BETAMETHASONE 0.1%+LEVOCETIRIZINE 2%+LIDOCAINE 5%+COLLAGENASE 350U+GM.

In Composition 2.3, the composition includes the following ingredients provided for in a base for topical application to a patient: FLUTICASONE 1%+LEVOCETIRIZINE 2%+LIDOCAINE 5%+GABAPENTIN 8%+TRAMADOL 5%

In Composition 2.4, the composition includes the following ingredients provided for in a base for topical application to a patient: FLUTICASONE 1%+LEVOCETIRIZINE 2%+LIDOCAINE 5%+TRANILAST 1%.

In Composition 2.5, the composition includes the following ingredients provided for in a base for topical application to a patient: TRANILAST 1%+BETAMETHASONE 0.1%+LEVOCERTIRIZINE 2%+GABAPENTIN 6%.

In Composition 2.6, the composition includes the following ingredients provided for in a base for topical application to a patient: FLUTICASONE 1%+LEVOCETIRIZINE 2%.

In Composition 2.7, the composition includes the following ingredients provided for in a base for topical application to a patient: FLUTICASONE 0.05%+LEVOCETIRIZINE 2%.

In Composition 2.8, the composition includes the following ingredients provided for in a base for topical application to a patient: SALICYLIC ACID 3%+RETINOIC ACID (TRETINOIN ACID) 0.1%+BETAMETHASONE 0.05%.

In Composition 2.9, the composition includes the following ingredients provided for in a base for topical application to a patient: SALICYLIC ACID 3%+RETINOIC ACID (TRETINOIN ACID) 0.1%+FLUTICASONE 1%+HYDROQUINONE 2%.

In Composition 3.0, the composition includes the following ingredients provided for in a base for topical application to a patient: BETAMETHASONE 0.1%+LEVOCETIRIZINE 2%.

In Composition 3.1, the composition includes the following ingredients provided for in a base for topical application to a patient: FLUTICASONE 1%+LEVOCETIRIZINE 2%+LIDOCAINE 5%+NIFEDIPINE 2%

In Composition 3.2, the composition includes the following ingredients provided for in a base for topical application to a patient: FLUTICASONE 1%+LEVOCETIRIZINE 2%+ZINC OXIDE 11%.

In Composition 3.3, the composition includes the following ingredients provided for in a base for topical application to a patient: BETAMETHASONE 0.1%+LEVOCETIRIZINE 2%+LIDOCAINE 5%+UREA 40%.

In Composition 3.4, the composition includes the following ingredients provided for in a base for topical application to a patient: BETAMETHASONE 0.1%+LEVOCETIRIZINE 2%+LIDOCAINE 5%+GABAPENTIN 6%.

In Composition 3.5, the composition includes the following ingredients provided for in a base for topical application to a patient: BETAMETHASONE 0.1%+LEVOCETIRIZINE 2%+LIDOCAINE 5%+VERAPAMIL 10%.

In Composition 3.6, the composition includes the following ingredients provided for in a base for topical application to a patient: LEVOCETIRIZINE 2%+LIDOCAINE 5%+UREA 40%.

In Composition 3.7, the composition includes the following ingredients provided for in a base for topical application to a patient: BETAMETHASONE 0.1%+LEVOCETIRIZINE 2%+EMU OIL 2%.

In Composition 3.8, the composition includes the following ingredients provided for in a base for topical application to a patient: TRANILAST 1%+BETAMETHASONE 0.1%+LEVOCERTIRIZINE 2%+IMIQUIMOD 2.5%.

In Composition 3.9, the composition includes the following ingredients provided for in a base for topical application to a patient: FLUTICASONE 1%+LEVOCETIRIZINE 2%+LIDOCAINE 5%+VERAPAMIL 5%.

In Composition 4.0, the composition includes the following ingredients provided for in a base for topical application to a patient: TRANILAST 1%+FLUTICASONE 1%+LEVOCERTIRIZINE 2%+KETOPROFEN 5%.

In Composition 4.1, the composition includes the following ingredients provided for in a base for topical application to a patient: FLUTICASONE 1%+UREA 40%+SALICYLIC ACID 30%.

In Composition 4.2, the composition includes the following ingredients provided for in a base for topical application to a patient: IMIQUIMOD 2.5%+FLUTICASONE 1%+LEVOCETIRIZINE 2%+LIDOCAINE 5%.

In Composition 4.3, the composition includes the following ingredients provided for in a base for topical application to a patient: FLUTICASONE 1%+LEVOCETIRIZINE 2%+LIDOCAINE 5%+GABAPENTIN 6%.

In Composition 4.4, the composition includes the following ingredients provided for in a base for topical application to a patient: TRANILAST 1%+CAFFEINE 1%+PENTOXIFYLLINE 1%.

In Composition 4.5, the composition includes the following ingredients provided for in a base for topical application to a patient: BETAMETHASONE 0.05%+UREA 40% IN VERSABASE.

In Composition 4.6, the composition includes the following ingredients provided for in a base for topical application to a patient: SALICYLIC ACID 3%+RETINOIC ACID (TRETINOIN ACID) 0.1%.

In Composition 4.7, the composition includes the following ingredients provided for in a base for topical application to a patient: IMIQUIMOD 1%+BETAMETHASONE 0.1%+LEVOCETIRIZINE 2%+LIDOCAINE 5%.

In Composition 4.8, the composition includes the following ingredients provided for in a base for topical application to a patient: LEVOCETIRIZINE 2%+VITAMIN E ACETATE 2%+EMU OIL 2%+TRETINOIN 0.005%.

In Composition 4.9, the composition includes the following ingredients provided for in a base for topical application to a patient: FLUTICASONE 0.25%.

In Composition 5.0, the composition includes the following ingredients provided for in a base for topical application to a patient: FLUTICASONE 2%+LEVOCETIRIZINE 2%+LIDOCAINE 5%.

In Composition 5.1, the composition includes the following ingredients provided for in a base for topical application to a patient: FLUTICASONE 0.25%+LEVOCETIRIZINE 2%+COLLAGENASE 350U+GM.

In Composition 5.2, the composition includes the following ingredients provided for in a base for topical application to a patient: TRANILAST 1%+FLUTICASONE 1%+LEVOCERTIRIZINE 2%+HYDROQUINONE 6%.

In Composition 5.3, the composition includes the following ingredients provided for in a base for topical application to a patient: IMIQUIMOD 2.5%+FLUTICASONE 1%+LEVOCETIRIZINE 2%+LIDOCAINE 5%+TRAMADOL 5%.

In Composition 5.4, the composition includes the following ingredients provided for in a base for topical application to a patient: FLUTICASONE 1%+LEVOCETIRIZINE 2%+LIDOCAINE 5%+VERAPAMIL 10%.

In Composition 5.5, the composition includes the following ingredients provided for in a base for topical application to a patient: BETAMETHASONE 0.1%+IMIQUIMOD 2.5%+PHENYTOIN 5%.

In Composition 5.6, the composition includes the following ingredients provided for in a base for topical application to a patient: RETINOIC ACID 0.05%+BENZOYL PEROXIDE 5%+BETAMETHASONE 0.1%+SALICYLIC ACID 3% IN PRACASIL.

In Composition 5.7, the composition includes the following ingredients provided for in a base for topical application to a patient: IMIQUIMOD 2.5%+BETAMETHASONE 0.1%+LEVOCETIRIZINE 2%.

In Composition 5.8, the composition includes the following ingredients provided for in a base for topical application to a patient: TRANILAST 1%+FLUTICASONE 1%+LEVOCERTIRIZINE 2%+PENTOXIFYLLINE 0.5%.

In Composition 5.9, the composition includes the following ingredients provided for in a base for topical application to a patient: FLUTICASONE 1%+LEVOCETIRIZINE 2%+LIDOCAINE 5%+HYDROQUINONE 5%.

In Composition 6.0, the composition includes the following ingredients provided for in a base for topical application to a patient: FLUTICASONE 1%+LEVOCETIRIZINE 2%.

In Composition 6.1, the composition includes the following ingredients provided for in a base for topical application to a patient: IMIQUIMOD 2.5%+FLUTICASONE 1%+LEVOCETIRIZINE 2%.

In Composition 6.2, the composition includes the following ingredients provided for in a base for topical application to a patient: TRANILAST 1%+BETAMETHASONE 0.1%+LEVOCERTIRIZINE 2%+PENTOXIFYLLINE 0.5%.

In Composition 6.3, the composition includes the following ingredients provided for in a base for topical application to a patient: TRANILAST 1%+HYDROCORTISONE 2.5%+LEVOCERTIRIZINE 2%.

In Composition 6.4, the composition includes the following ingredients provided for in a base for topical application to a patient: FLUTICASONE 1%+LEVOCETIRIZINE 2%+LIDOCAINE 5%+PENTOXYFYLLINE 5%.

In Composition 6.5, the composition includes the following ingredients provided for in a base for topical application to a patient: LEVOCETIRIZINE 2%+VITAMIN E ACETATE 2%+EMU OIL 2%+TRETINOIN 0.05%.

In Composition 6.6, the composition includes the following ingredients provided for in a base for topical application to a patient: TRANILAST 1%+FLUTICASONE 1%+LEVOCERTIRIZINE 2%+PENTOXIFYLLINE 5%

In Composition 6.7, the composition includes the following ingredients provided for in a base for topical application to a patient: IMIQUIMOD 2.5%+FLUTICASONE 1%+LEVOCETIRIZINE 2%+LIDOCAINE 5%+PENTOXIFYLLINE 5%.

In Composition 6.8, the composition includes the following ingredients provided for in a base for topical application to a patient: SALICYLIC ACID 3%+RETINOIC ACID (TRETINOIN) 0.05%+FLUTICASONE 0.5%.

In Composition 6.9, the composition includes the following ingredients provided for in a base for topical application to a patient: FLUTICASONE 1%+FLUCONAZOLE 1%+LIDOCAINE 5%.

In Composition 7.0, the composition includes the following ingredients provided for in a base for topical application to a patient: VITAMIN E ACETATE 2%+EMU OIL 2% IN PRACASIL.

In Composition 7.1, the composition includes the following ingredients provided for in a base for topical application to a patient: BETAMETHASONE 0.1%+LEVOCETIRIZINE 2%.

In Composition 7.2, the composition includes the following ingredients provided for in a base for topical application to a patient: BETAMETHASONE 0.1%+LEVOCETIRIZINE 2%+LIDOCAINE 5%+PENTOXIFYLLINE 2%.

In Composition 7.3, the composition includes the following ingredients provided for in a base for topical application to a patient: FLUTICASONE 1%+LEVOCETIRIZINE 2%+LIDOCAINE 5%+KETAMINE 5%+GABAPENTIN 6%.

In Composition 7.4, the composition includes the following ingredients provided for in a base for topical application to a patient: TRANILAST 1%+BETAMETHASONE 0.1%+LEVOCERTIRIZINE 2%+LIDOCAINE 2%.

In Composition 7.5, the composition includes the following ingredients provided for in a base for topical application to a patient: TRANILAST 2%+BETAMETHASONE 0.1%+LEVOCERTIRIZINE 2%+KOJIC ACID 2%.

In Composition 7.6, the composition includes the following ingredients provided for in a base for topical application to a patient: BETAMETHASONE 0.1% IN PRACASIL GEL.

In Composition 7.7, the composition includes the following ingredients provided for in a base for topical application to a patient: LEVOCETIRIZINE 2%+EMU OIL 2%+TRETINOIN 0.1%.

In Composition 7.8, the composition includes the following ingredients provided for in a base for topical application to a patient: BETAMETHASONE 0.1%+LEVOCETIRIZINE 2%+SODIUM HYALURONATE 2%+LIDOCAINE 5%.

In Composition 7.9, the composition includes the following ingredients provided for in a base for topical application to a patient: TRANILAST 1%+BETAMETHASONE 0.5%+LEVOCERTIRIZINE 1%

In Composition 8.0, the composition includes the following ingredients provided for in a base for topical application to a patient: FLUTICASONE 1%+LEVOCETIRIZINE 2%+LIDOCAINE 5%+HYDROQUINONE 4%.

In Composition 8.1, the composition includes the following ingredients provided for in a base for topical application to a patient: LEVOCETIRIZINE 2%.

In Composition 8.2, the composition includes the following ingredients provided for in a base for topical application to a patient: IMIQUIMOD 2.5%+BETAMETHASONE 0.1%+LEVOCETIRIZINE 2%+LIDOCAINE 5%+PENTOXIFYLLINE 1%.

In Composition 8.3, the composition includes the following ingredients provided for in a base for topical application to a patient: FLUTICASONE 1%+LEVOCETIRIZINE 2%+LIDOCAINE 5%+KETAMINE 5%

In Composition 8.4, the composition includes the following ingredients provided for in a base for topical application to a patient: BETAMETHASONE 0.1%+LEVOCETIRIZINE 2%+LIDOCAINE 5%+HYDROQUINONE 4%.

In Composition 8.5, the composition includes the following ingredients provided for in a base for topical application to a patient: IMIQUIMOD 0.1%+BETAMETHASONE 0.05% IN SILOMAC GEL.

In Composition 8.6, the composition includes the following ingredients provided for in a base for topical application to a patient: BETAMETHASONE 0.1%+LEVOCETIRIZINE 2%+PENTOXIFYLLINE 0.5%+PRILOCAINE 3%+GABAPENTIN 5%.

In Composition 8.7, the composition includes the following ingredients provided for in a base for topical application to a patient: DESOXIMETASONE 0.25%+UREA 40% IN PRACASIL.

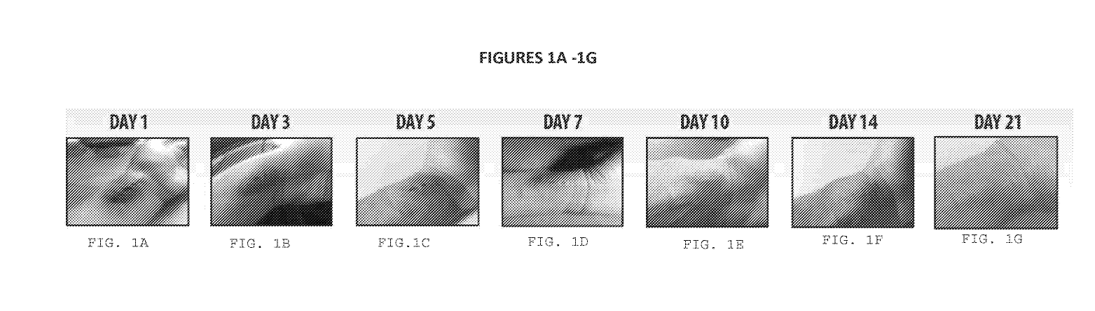

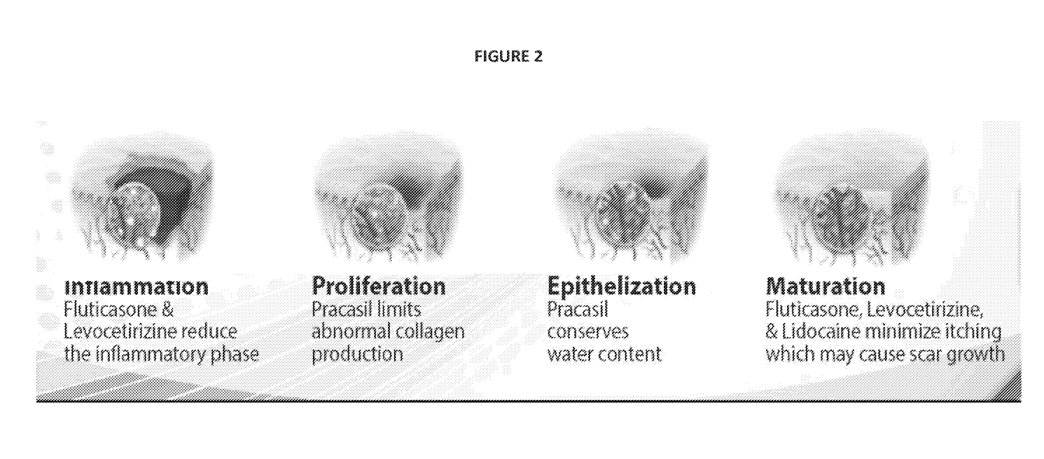

In a first case study a 51 year old female with type 1 diabetes had hand surgery to remove a ganglion cyst. The Patient was prescribed the Composition to be used twice daily. The compounded gel consisted of Fluticasone 1%, Levocetrizine 2%, and Lidocaine 5% made in a Pracasil base. FIGS. 1A-1G are pictures of the scar reduction over a three week period. FIG. 2 shows as an illustration the phases of treatment of a wound when using a composition in accordance with the present invention.

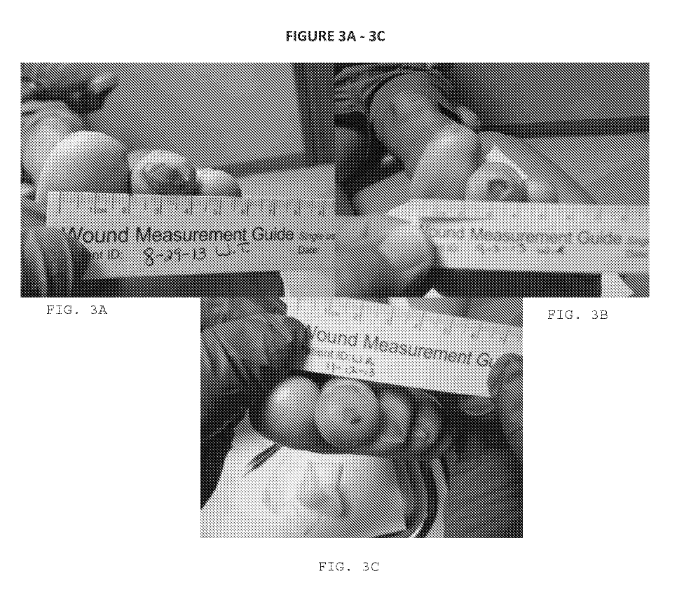

In a second case study, a 96 year old male presented with recent history of draining wound on the distal aspect of his 2nd toe. Toe is hammered with distal clavi. Past medical history includes Diabetes with Neuropathy, Hypertension and high Cholesterol. Current Medications include: Metanx, Lisinopril, Zyrtec, Lipitor, Citracel, Coumadin, Celebrex, Allopurinal, CoQ-10, and Fish Oil. The patient presented with Malodorus wound on the tip of 2nd toe left foot. During treatment, the wound was debrided, cultures taken, cleansed with Asept wound cleaner and the wound measured at 9×14 mm 2 mm in depth (FIG. 3A). The wound was debrided and patient started on Trinity antibiotic wound Gel, #10, to begin twice daily application of wound gel and an application of a non-adhesive dressing and/or dry sterile dressing to cover. As illustrated in FIGS. 3B and 3C the wound is measured and shown to significantly reduce during treatment. Patient Treatment included weekly debridement and continued with the Trinity Wound Gel follow up with Dry Sterile Dressing. The wound healed in 9 weeks. The patient was then taken to surgery and correction of hammertoe deformity was performed.

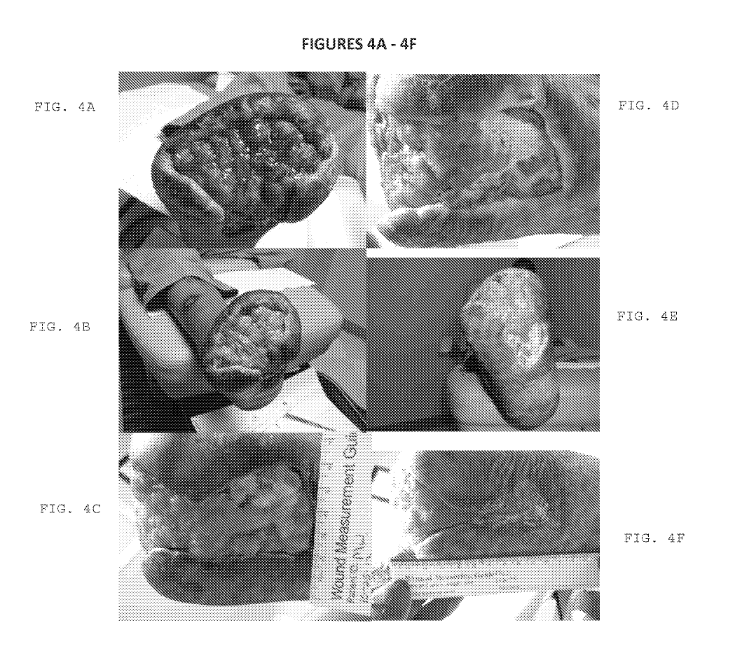

In a third case study (FIGS. 4A-4F) a patient is a 50 year old black male who was admitted for a diabetic foot infection. The patient is Insulin Dependent and developed black eschar on plantar aspect of left foot, sub 2nd metatarsal head and great toe. Multiple debridements and Incision and Drainage procedures were performed. Developed Deep Vein Thrombosis in left arm and lower extremity. A Transmetatarsal Amputation was performed and wound dehisced. Left lower extremity vascularity was non reconstructable and patient refused Below Knee Amputation. Patient presented to office post discharge from hospital for his diabetic foot infection. Initially presentation had been great toe and ball of foot and had progressed. Vascular Consult and Infectious Disease Consults with intravenous antibiotic Therapy, Incision and Drainage, multiple debridements and finally Transmet amputation. He is currently on blood thinner due to his Deep Vein Thrombosis. Wound dehisced with hematoma and necrotic tissue. Treatment-wound was debrided, hematoma evacuated, cultures taken, cleansed with Vasche wound wash then packed with ¼″ Iodoform guaze. f/u Dry Sterile Dressing. Trinity Wound Gel Rx with Tobromycin, Vancomycin, Mupirocin, Phenytoin, Misoprostol, Nifedipine, and Lidocaine. Patient was seen weekly for debridments and wound evaluation. On Aug. 22, 2013 the hematoma formation was almost resolved and pink granulation tissue was noted. We started utilizing Matristem grafts to augment our treatment protocol on Sep. 12, 2013. Wound is continuing to heal with twice daily application of Trinity Wound Gel. Initial wound measurements length-118 mm, distal width-93 mm, middle width-45 mm, proximal width-50 mm. Current length-98 mm, distal width-66 mm, middle width-3 mm, proximal width 15 mm.

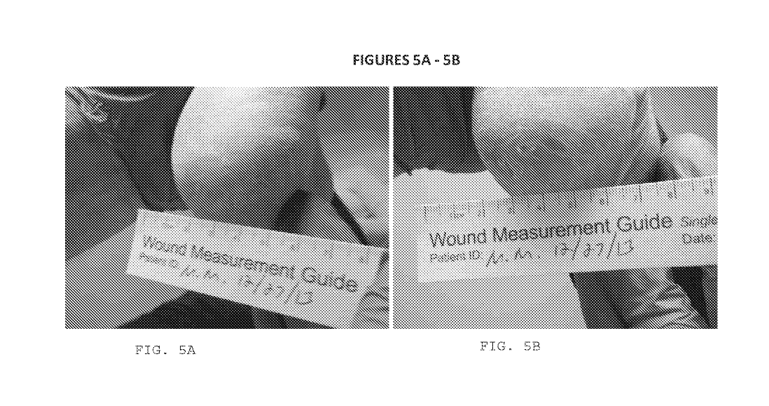

In a fourth case study, FIGS. 5A-5B, the patient is a 47 year old female, presenting with a wound on the posterior plantar aspect of her left heel. The wound has been present for several months duration. Past Medical History is positive for Insulin dependent Diabetes with Neuropathy, and hypothyroid. Current medications include Insulin both Lantus and Novolog, Gabapentin, Synthroid, and Lexapro. Initial office visit Dec. 5, 2013 the patient was presented with a wound Measuring 8×7 mm, 5 mm in depth, with some granulation tissue in base of wound. There was no erythema or pain associated with ulcer. Initial treatment included debridement, cleansing with Vashe wound wash, application of Matristem graft, covered with adaptic (steri-stripped in place) initially applied Ag(Silver) gel, follow up with dry sterile dressing. Trinity Wound Gel was ordered with Vancomycin and Mupirocin, to apply twice daily over adaptic and graft once received from Trinity. Patient follow up was weekly. The progress was measured as follows: Dec. 12, 2013 week 1—wound size 7×7 mm, 5 mm depth; Dec. 20, 2013 week 2—wound size 5×3 mm, 3 mm depth; and Dec. 27, 2013 week 3—Healed. In conclusion the wound healed completely in 21 days of treatment with treatment above. Only one graft application on her initial office visit, then weekly debridement.

From the foregoing and as mentioned above, it will be observed that numerous variations and modifications may be effected without departing from the spirit and scope of the novel concept of the invention. It is to be understood that no limitation with respect to the specific embodiments illustrated herein is intended or should be inferred. It is, of course, intended to cover by the appended claims all such modifications as fall within the scope of the claims.

We claim:

1. A wound healing and/or scar reduction composition comprising a base compound in combination with the following active ingredients, Phenytoin, Misoprostol, Nifedipine, and Lidocaine.

2. The Composition of claim 1, wherein the active ingredients are provided in the following percentages based on a finished composition: Phenytoin: 1-10%, Misoprostol: 0.001-0.003%, Nifedipine: 1-5%, and Lidocaine: 1-6%.

3. The Composition of claim 2 further comprising one or more of the following ingredients:
Gentamicin: 0.5-2.0%, Tobramicin: 0.5-2.0%, Ciprofloxacin: 0.5-3.0%, Levofloxacin: 0.5-5.0%, Vancomycin: 2-6%, Linezolid: 0.5-5.0%, Mupirocin: 1-5%, Clindamycin: 0.5-5.0%, Doxycycline: 0.5-5.0%, Amikacin: 0.5-3.0%, Cefuroxime: 1-3%, Meropenem: 0.5-3.0%, Metronidazole: 0.5-3.0%, Bacitracin: 100-1000 U/GM, Colistimethate: 0.5-3.0%, Ampicillin: 0.5-3.0%, and Tetracycline: 0.5-3.0%.

4. The Composition of claim 3 or 2 further comprising one or more of the following ingredients: Itraconazole: 0.5-3.0%, Fluconazole: 0.5-3.0%, and Nystatin: 10,000-50,000 U/GM.

5. The Composition of claim 4 further comprising one of more of the following ingredients:
Xylitol: 0.5-2.0%, Rifampin: 0.5-5.0%, Collagen: 0.5-5.0%, Collagenase: 100-500 U/GM, Fluticasone: 0.1-2.0%, Betamethasone: 0.025-0.2%, and Urea: 5-50%.

6. The Composition of claim 3 or 2 further comprising one of more of the following ingredients: Xylitol: 0.5-2.0%, Rifampin: 0.5-5.0%, Collagen: 0.5-5.0%, Collagenase: 100-500 U/GM, Fluticasone: 0.1-2.0%, Betamethasone: 0.025-0.2%, and Urea: 5-50%.

7. The Composition of claim 1 further comprising Amazonian oil and Pracaxi oil.

8. The Composition of claim 1 further comprising Levocetirizine, Pentoxyifylline, Gabapentin, Tranilast, Vitamin E, Emu Oil, Salicylic Acid, Tretinoin (Retinoic Acid).

9. A scar reduction composition, comprising: Fluticasone Propionate, Levocetirizine, Lidocaine.

10. The composition of claim 9 wherein the active ingredients are provided in the following percentages based on a finished composition: Fluticasone Propionate 1%, Levocetirizine 2%, Lidocaine 5%, in base gel for topical application to a scar and/or skin wound.

11. A scar reduction composition, comprising: Imiquimod, Fluticasone Propionate, Levocetirizine, Lidocaine.

12. The composition of claim 11 wherein the active ingredients are provided in the following percentages based on a finished composition: Imiquimod 2.5%, Fluticasone Propionate 1%, Levocetirizine 2%, Lidocaine 5%, in a base gel for topical application to a scar.

13. A scar reduction composition, comprising: Imiquimod, Betamethasone Dipropionate, Pentoxifylline.

14. The composition of claim 11 wherein the active ingredients are provided in the following percentages based on a finished composition: Imiquimod 2.5%, Betamethasone Dipropionate 0.1%, Pentoxifylline 3%, in a base gel for topical application to a scar.

15. A composition for reducing stretch marks, comprising: Levocetirizine, Vitamin E Acetate, Emu Oil.

16. The composition of claim 15 wherein the active ingredients are provided in the following percentages based on a finished composition: Levocetirizine 2%, Vitamin E Acetate 2%, Emu Oil 2%, in base gel for topical application.
